(19) United States
(12) Patent Application Publication
(10) Pub. No.: US 2025/0281334 A1
(43) Pub. Date: Sep. 11, 2025

(54) Sterile Non-Adherent Contoured Dressing/Bandage for Breast Cancer Radiation Treatment

(71) Applicant: Darnella Louise Banks, Englewood, NJ (US)

(72) Inventor: Darnella Louise Banks, Englewood, NJ (US)

(21) Appl. No.: 18/954,792

(22) Filed: Nov. 21, 2024

(51) Int. Cl. A61F 13/0246; A61L 15/22; A61L 15/58

(52) U.S. Cl. CPC A61F 13/0253; A61L 15/225; A61L 15/58

(57) ABSTRACT

The present invention relates to the improvement of dressings and bandages specifically used for wound care of the breast and underarm post cancer radiation treatment, and general wounds and surgeries affecting the breast. There are known dressings that are limited to square and rectangular shapes, requiring multiple pieces to cover the breast and underarm. There are also known bandages, such as rolled gauze, that require wrapping the entire torso and/or across the shoulders and back to secure the dressings. However, this present invention is a single dressing contoured to the shape of the breast with a top, bottom and sides that extend beyond the affected breast and underarm area. This invention is also a single bandage that is backless, only covering the front and sides of the body, with edges that extend beyond the affected area and lined along the back with silicon that adheres to the unaffected skin.

### TECHNICAL FIELD

The present invention relates to a specialized non-adherent dressing/bandage designed to address the unique needs of individuals undergoing breast cancer radiation treatment, wherein the dressing is contoured in the shape of the female breast, with extended edges to provide comprehensive coverage and protection to the affected area.

### BACKGROUND ART

Usually, patients protect treatment areas from rubbing, pressure or irritation by wearing loose, soft clothing next to the skin. Cotton and silk are less irritating on radiated skin than harsh fabrics like wool and denim. Ask your radiation therapy team before you use anything on the skin in the treatment area.

Some prior art recommends use a dressing on your skin that has broken down (become inflamed and started peeling and become moist) due to your radiotherapy or proton beam therapy treatment. Usually a soft, absorbent dressing that is very comfortable to wear. It cleans the wound, moisturizes the skin, reduces inflammation and helps reduce pain. The dressings help to protect the treated area, absorb any oozing from the wound, keep your skin moist, which helps promote healing, keep the area clean. It may be helpful to share this factsheet with your family, district or practice nurse, if you have to see them to have your dressings changed.

Some instructions for use include wash and dry your hands well, before and after applying the dressing. Make sure to have a clean space (e.g. a clean towel) on which to place the fresh products, and to have everything you need to do the cleansing and dressing change. If you are wearing a dressing that must be removed, remove the old dressing and place it in the bin. Gently lift the border on the dressing and slowly pull back the dressing until fully removed. Your skin may bleed a little after cleansing. This is normal. The dressing may need to be cut to the required shape and size. When changing the dressing you may, or may not, need to clean the wound as it contains wound cleaning substances.

However, the above prior art does not disclose features being taught in the present.

### OBJECT OF THE INVENTION

The main object of the present invention is to provide a single non-adherent dressing/bandage for breast cancer radiation treatment.

The following summary is an explanation of some of the general inventive steps for the dressing.

### SUMMARY OF INVENTION

The present invention describes a single non-adherent dressing/bandage for breast cancer radiation treatment is specifically contoured and molded to fit the shape of the female breast. Its extended edges ensure coverage beyond the affected area, safeguarding against the side effects of radiation treatment. The dressing's innovative design, along with its optional materials and adhesive components, provides maximum comfort and protection during the healing process.

The above summary is not an extensive overview of the invention and does not intend to limit the scope beyond what is described and claimed as a summary.

### BRIEF DESCRIPTION OF FIGURES

The novel features believed to be characteristic of the illustrative embodiments are outlined in the appended claims. The illustrative embodiments, however, as well as a preferred mode of use, further objectives, and descriptions thereof, will best be understood by reference to the following detailed description of one or more illustrative embodiments of the dressings when read in conjunction with the accompanying drawings.

FIG. 1 is an illustration of a frontal view of the invention for left breast.

FIG. 2 is an illustration of a side view of the invention for left breast.

FIG. 3 is an illustration of a frontal view of the invention for both breasts.

FIG. 4 is an illustration of a frontal view of the invention for right breast.

FIG. 5 is an illustration of a side view of the invention for right breast.

The above figures describe the components in the accessory, their layout, and their location. The drawings identify the various parts and also the location of each compartment of the mat. The above drawings depict the complete standard accessory location sections. Customization of the mat to an equipment specific model is possible.

### DETAILED DESCRIPTION OF INVENTION

In the following detailed description of the invention, numerous details, examples, and embodiments of the invention are described. However, it will be clear and apparent to those knowledgeable in the field that the invention is not limited to the embodiments set forth and that the invention can be adapted for a wider range of applications.

In this description, the terms “comprising,” “including,” “containing,” “characterized by,” and grammatical equivalents thereof are inclusive or open-ended terms that do not exclude additional, un-recited elements or method acts, but also include the more restrictive terms “consisting of” and “consisting essentially of” and grammatical equivalents thereof. As used herein, the term “may” with respect to a material, structure, feature or method act indicates that such is contemplated for use in implementation of an embodiment of the disclosure and such term is used in preference to the more restrictive term “is” so as to avoid any implication that other, compatible materials, structures, features and methods usable in combination therewith should or must be, excluded.

The singular forms “a,” “an,” and “the” are intended to include the plural forms as well, unless the context clearly indicates otherwise. The term “and/or” includes any and all combinations of one or more of the associated listed items. As used herein, relational terms, such as “first,” “second,” “top,” “bottom,” “upper,” “lower,” “over,” “under,” etc., are used for clarity and convenience in understanding the disclosure and do not connote or depend on any specific preference, orientation, or order, except where the context clearly indicates otherwise.

In the present invention, a dressing/bandage is contoured in a shape that mimics the female breast to provide optimal coverage and protection. Its non-adherent properties prevent sticking to the dry secretions of the wound, thereby facilitating the healing process. The extended size and shape of the dressing offer protection from external factors such as clothing friction and arm movement.

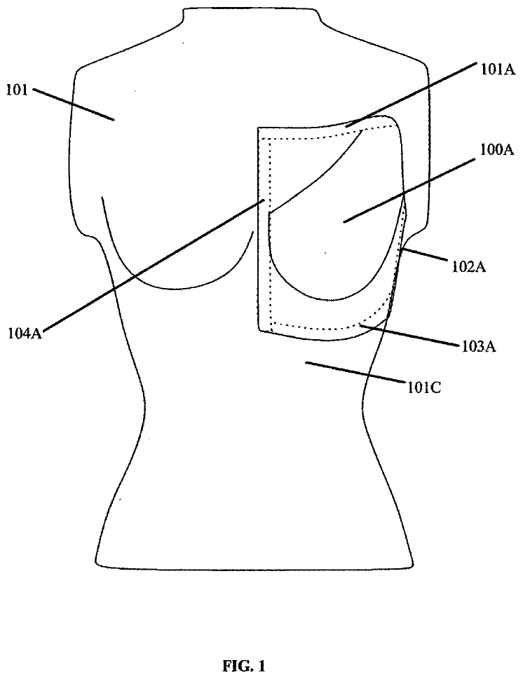

FIG. 1 presents an illustration of a frontal view of the invention for left breast. The top chest 105 from side to side is 8 inches. The bust (the fullest part of breast) to back is 9 inches, bust to breastbone is 5 inches, bust to chest is 6 inches, front bust to stomach is 5 inches and chest to stomach (down across front bust): 11 inches. Part 101A illustrates the silicon adhesive on the back side of the invention to adhere to the skin 101B on the left side 102, lower side 103A and on the right side 104 as described in the present invention.

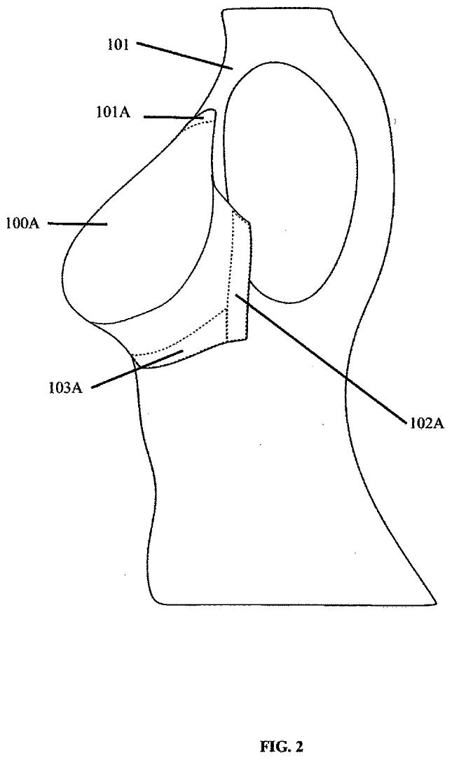

FIG. 2 is an illustration of a side view of the invention for left breast (not shown). Underarm top to bottom is 5 inches, chest to stomach (down the side of breast) is 8 inches, bust to chest is 6 inches and bust to back is 9 inches. Part 101A illustrates the silicon adhesive on the back side 103A of the invention to adhere to the skin as well as on the left side 102 and lower side 103A.

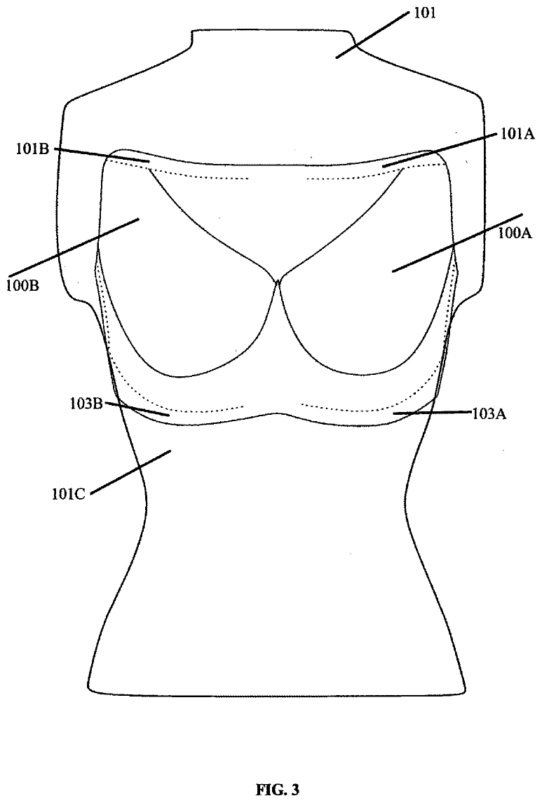

FIG. 3 is an illustration of a frontal view of the invention for both breasts. The top chest from side to side is 16 inches. The front of the bust is 8 inches. Left side to right side is 26 inches. Bust to stomach: 5 inches, bust to top chest: 6 inches, and the chest to stomach: 11 inches. Part 101A illustrates the silicon adhesive on the back side of the invention to adhere to the skin on the back upper left side 104, back upper right side 101A, lower right side 103A and lower right side 103B.

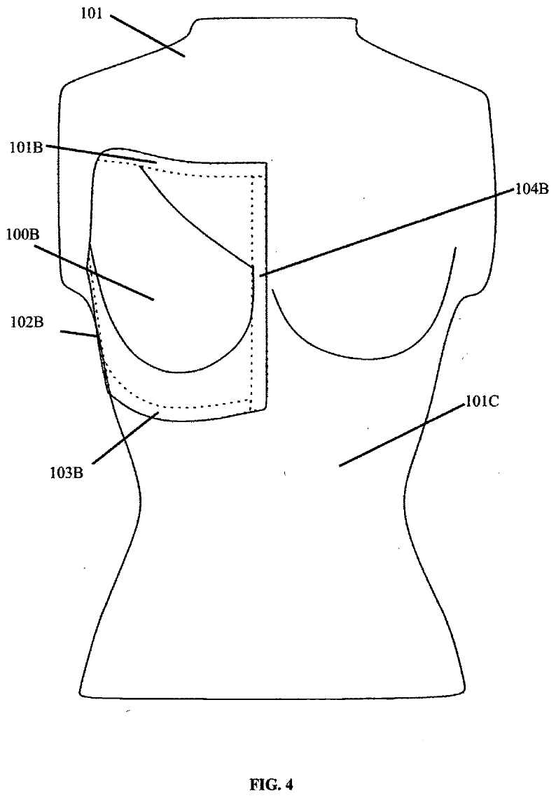

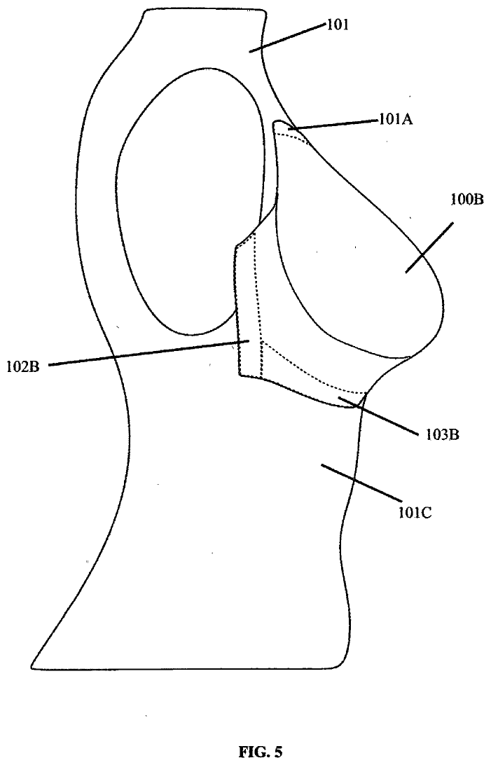

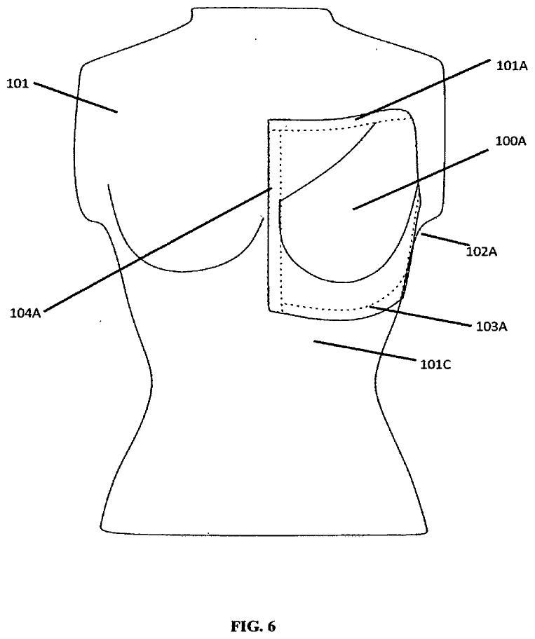

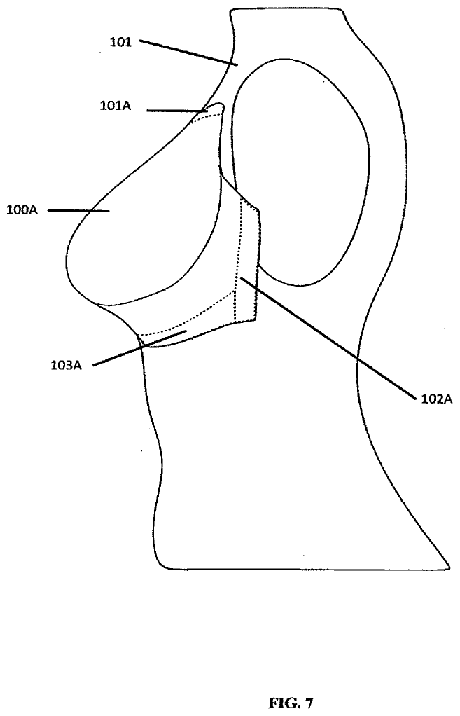

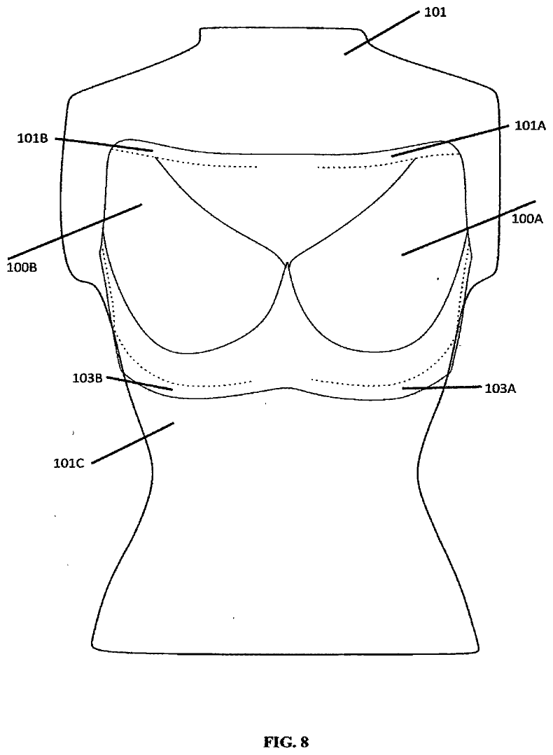

FIG. 4 is an illustration of a frontal view of the invention for right breast. The top chest 105 from side to side is 8 inches. The bust (the fullest part of breast) to back is 9 inches, bust to breastbone is 5 inches, bust to chest is 6 inches, front bust to stomach is 5 inches and chest to stomach (down across front bust): 11 inches. Part 102 illustrates the silicon adhesive on the left back side 104 of the invention to adhere to the skin and also designed to cover middle chest 102 and lower right side 102B as shown.

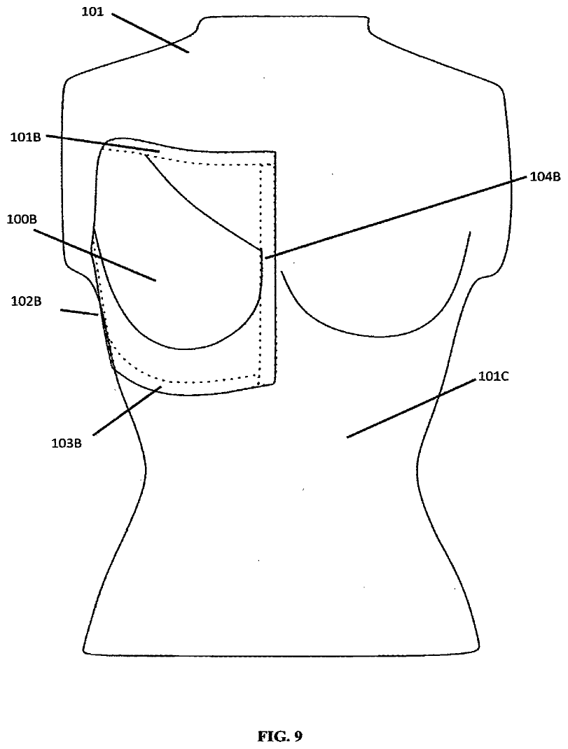

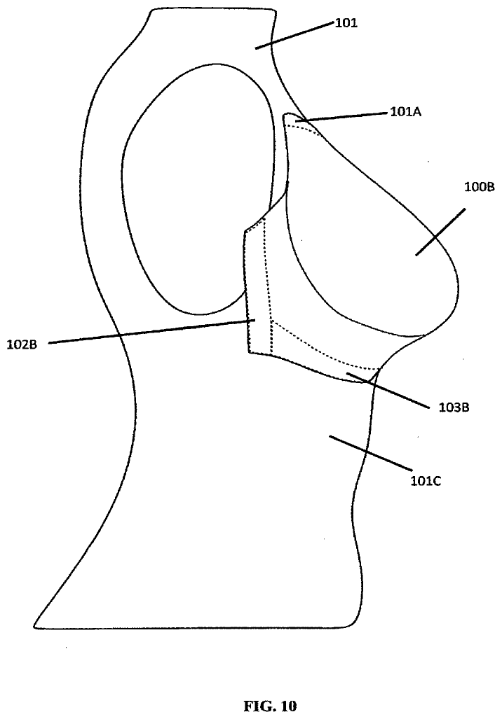

FIG. 5 is an illustration of a side view of the invention for right breast (not shown). Underarm top to bottom 103B is 5 inches, chest to stomach (down the side of breast 103B) is 8 inches, bust to chest is 6 inches and bust to back is 9 inches. Part 101A illustrates the silicon adhesive on the back side of the invention to adhere to the skin including right middle side 104 of the breast. The overall measurements increase and decrease by 1 inch based on the bandage size and cup size.

### Product Options:

The dressing/bandage is available in three models, catering to both single and double breast requirements. FIG. 1—front, FIG. 1—back and FIG. 2—front, FIG. 2—back, are designed for single-breast applications, covering the left and right breast, respectively. FIG. 3—front, FIG. 3—side, FIG. 3—back, is suitable for both breasts. Each model is available in various sizes, based on standard bra cup sizes. Part 101A of FIG. 1—back. FIG. 2—back and FIG. 3—back, shows the silicon lining on the edge of the dressing/bandage that adheres to the unaffected areas of the skin.

### Optional Materials:

The dressing/bandage can be manufactured using a range of non-adherent materials to provide comfort and support during the healing process. These materials include lite hydro cellular foam, non-adherent silicone, polyester, rayon, cotton, polyester blend, and nylon. Additionally, these materials can be pre-treated with petroleum or other healing creams and ointments to facilitate the recovery process.

Attachment: The back of the dressing/bandage incorporates silicone gel adhesive around its edges, facilitating easy application and attachment to the unaffected skin beyond the affected areas. The adhesive is protected and covered by removable coated paper, which can be easily peeled off before application.

While the invention has been described with reference to numerous specific material, one of ordinary knowledge in the field of dressings will recognize that the invention can be embodied in other specific forms without departing from the spirit of the invention. Specific adhesives and operations may not be performed in one continuous series, and different specific methods may be performed in different embodiments. Furthermore, the processes or methods could be implemented using several sub-processes, or as part of adhesive process. Thus, one of ordinary knowledge in the field would understand that the invention is not to be limited by the foregoing illustrative details, but rather is to be defined by the appended claims.

### INDUSTRIAL APPLICATIONS

The primary applications of the single non-adherent dressing/bandage include breast cancer radiation treatment, post-operative care following breast surgery, and use in both cosmetic and medical procedures. This invention will secure the unique functionalities of the non-adherent dressing/bandage, ensuring the protection and comfort of individuals undergoing breast cancer treatment and recovery and other treatments affecting the breast.

The invention claimed is:

1. A non-adherent dressing/bandage for breast radiation treatment, molded in the shape of the female breast, with extended edges at the top, bottom and sides for protection and adherence beyond the radiation-affected area.

2. The dressing/bandage of claim 1, further comprising silicone gel adhesive along its top rim, both sides, and along the bottom.

3. The dressing/bandage of claim 1, wherein the bandage is available in various sizes based on standard bra cup sizes.

4. The dressing/bandage of claim 1, wherein the bandage is constructed using non-adherent materials.

5. The dressing/bandage of claim 4, wherein the non-adherent materials include Lite Hydro-cellular Foam, non-adherent silicone, polyester and rayon, cotton and polyester blend, and nylon and polyester blend.
